(19) United States
(12) Patent Application Publication
(10) Pub. No.: US 2024/0261487 A1
(43) Pub. Date: Aug. 8, 2024

(54) Systems And Methods For Using Microfluidic Devices With Apheresis Systems

(71) Applicant: Terumo BCT, Inc., Lakewood, CO (US)

(72) Inventor: Thomas J. FELT, Boulder, CO (US)

(21) Appl. No.: 18/636,577

(22) Filed: Apr. 16, 2024

Related U.S. Application Data
(63) Continuation of application No. 17/977,626, filed on Oct. 31, 2022.
(60) Provisional application No. 63/274,883, filed on Nov. 2, 2021.

(51) Int. Cl. A61M 1/36; A61M 1/30

(52) U.S. Cl. CPC A61M 1/3695; A61M 1/304; A61M 2205/3337; A61M 2205/3362

(57) ABSTRACT

The present disclosure provides a system for using an in-line processing device with an apheresis device. The system includes a pressure system that includes a container configured to receive cells collected using the apheresis device and is configured to change a pressure of the container so as to direct the cells collected using the apheresis device to the in-line processing device.

### CROSS-REFERENCE TO RELATED APPLICATIONS

This application is a continuation of U.S. patent application Ser. No. 17/977,626 filed on Oct. 31, 2022, which claims the benefit of U.S. Provisional Application No. 63/274,883 filed on Nov. 2, 2021. The entire disclosures of the above applications are incorporated herein by reference.

### FIELD

The present disclosure relates to methods for using microfluidic devices, and more particularly, to systems for using in-line microfluidic devices with apheresis systems for cell processing.

### BACKGROUND

This section provides background information related to the present disclosure which is not necessarily prior art.

Apheresis includes extracting whole blood from a donor subject while the donor subject is connected to a specialized machine. The specialized machine may be configured to direct the whole blood through various tubes or channels to separate the whole blood into various components (or constituents). For example, in various aspects, plasma, red blood cells, white blood cells, and/or platelets may be separated from the whole blood using apheresis processes. The machine may be configured to separate and collect the plasma (and/or another desired component) in a bag or bottle for later use, including for different therapies, treatments, and/or transfusions. After the collection, the machine may be configured to return the remaining components of the whole blood to the donor subject.

### SUMMARY

This section provides a general summary of the disclosure, and is not a comprehensive disclosure of its full scope or all of its features.

In various aspects, the present disclosure provides a system for using an in-line processing device with an apheresis device. The system may include a pressure system. The pressure system may include a container configured to receive cells collected using the apheresis device and may be configured to change a pressure of the container so as to direct the cells collected using the apheresis device to the in-line processing device.

In one aspect, the pressure system may include a stationary plate and one or more moveable plates that are configured to move relative to the stationary plate. The container may be disposed between the stationary plate and the one or more moveable plates and movement of the one or more moveable plates may change the pressure of the container.

In one aspect, the pressure system may further include one or more motors configured to move the one or more moveable plates relative to the stationary plate.

In one aspect, the system may further include a fluid line that fluidly connects the apheresis device and the container. The fluid line may include a valve.

In one aspect, the fluid line may be a first fluid line, the valve may be a first valve, and the system may further include a second fluid line that fluidly connects the pressure device and the in-line processing device. The second fluid line may have a second valve.

In one aspect, the container may have two or more distinct compartments. A first compartment of the two or more distinct compartments may be configured to receive the cells collected using the apheresis device, and a second compartment of the two or more distinct compartments may be configured to receive a buffer solution.

In one aspect, the in-line processing device may be a microfluidic device.

In various aspects, the present disclosure provides a system for using a microfluidic device with an apheresis device. The system may include a pressure system that includes a container having two or more distinct compartments. A first compartment of the two or more distinct compartments may be configured to receive cells collected using the apheresis device. A second compartment of the two or more distinct compartments may be configured to receive a buffer solution. The pressure system may be configured to change a first pressure of the first compartment and a second pressure of the second compartment and to direct the cells collected using the apheresis device and the buffer solution to the microfluidic device.

In one aspect, the pressure system may include a stationary plate and one or more moveable plates that are configured to move relative to the stationary plate. The container may be disposed between the stationary plate and the one or more moveable plates and movement of the one or more moveable plates may change the first pressure of the first compartment and the second pressure of the second compartment.

In one aspect, the pressure system may further include one or more motors configured to move the one or more moveable plates relative to the stationary plate.

In one aspect, the one or more moveable plates may include a first plate aligned with the first compartment and a second plate aligned with the second compartment.

In one aspect, the pressure system may further include a first motor configured to move the first plate relative to the stationary plate, and a second motor configured to move the second plate relative to the stationary plate.

In one aspect, the system may further include a first fluid line that fluidly connects the first compartment and the apheresis device and a second fluid line that fluidly connects the second compartment and a buffer source. The first fluid line may include a first valve. The second fluid line may include a second valve.

In one aspect, the system may further include a third fluid line that fluidly connects the first compartment and a first port of the microfluidic device, and a fourth fluid line that fluidly connects the second compartment and a second port of the microfluidic device. The third fluid line may include a third valve. The fourth fluid line may include a fourth valve.

In various aspects, a method for processing cells collected using an apheresis device. The method may include receiving in a container the cells collected using the apheresis device, adjusting a pressure of the container, and at the adjusted pressure, transferring at least a portion of the cells collected using the apheresis device held by the container to a microfluidic device for processing.

In one aspect, the adjusting of the pressure may include decreasing the pressure inside the container from a first pressure to a second pressure.

In one aspect, the adjusting of the pressure may include increasing the pressure inside the consider form a first pressure to a second pressure.

In one aspect, the first pressure may be atmospheric pressure, and the second pressure may be about 70 pounds per square inch (psi).

In one aspect, the pressure may be adjusted using a pressure system that is fluidly coupled to the apheresis device and the microfluid device. The pressure system may include a stationary plate and one or more moveable plates that are configured to move relative to the stationary plate. The container may be disposed between the stationary plate and the one or more moveable plates and movement of the one or more moveable plates may change the pressure of the container.

In one aspect, the container may include a first compartment and a second compartment and the pressure system may further include a first fluid line that fluidly connects the first compartment and the apheresis device, a second fluid line that fluidly connects the second compartment and a buffer course, a third fluid line that fluidly connects the first compartment and the microfluidic device, and a fourth fluid line that fluidly connects the second compartment and the microfluidic device. The first fluid line may include a first valve. The second fluid line may include a second valve. The third fluid line may include a third valve. The fourth fluid line may include a fourth valve. The method may include closing the first and second valves and opening the third and fourth valves prior to the adjusting of the pressure.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a schematic of an example system for using a microfluidic device with an apheresis system, without requiring modifications to the apheresis system, in accordance with various aspects of the present disclosure; and

FIG. 2 is a cross-sectional illustration of an example pressure device in fluid communication with a microfluidic device and an apheresis system in accordance with various aspects of the present disclosure.

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers, and/or sections, these elements, components, regions, layers, and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

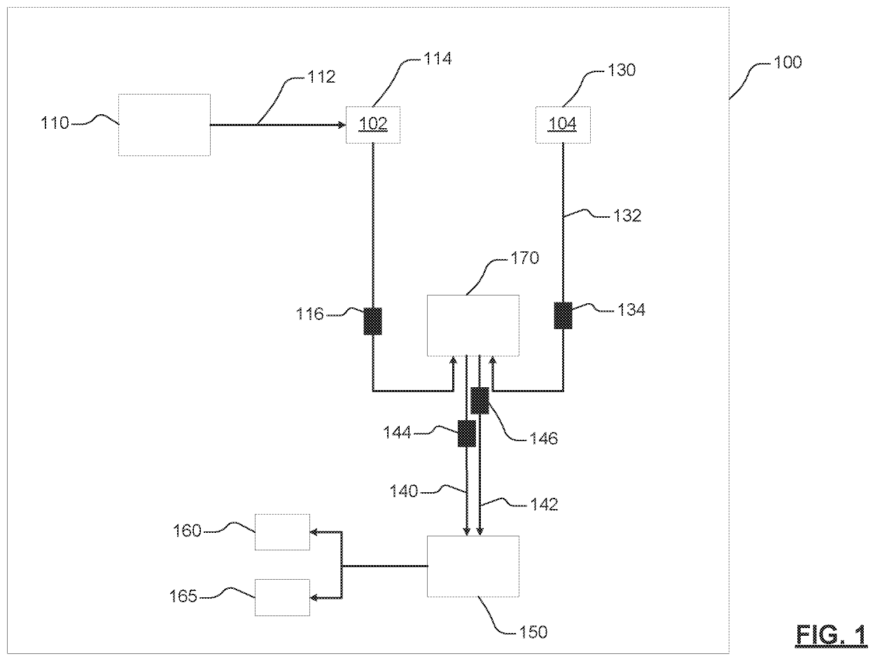

Systems and methods for using microfluidic devices, and more particularly, systems and methods for using in-line microfluidic devices with apheresis systems for cell processing are provided. For example, FIG. 1 is a schematic of an example system 100 for using a microfluidic device 150 with an apheresis system 110, without requiring modifications to the apheresis system 100 and/or the microfluidic device 150. The apheresis system 110 is configured to extract whole blood from a donor subject and to treat or collect a component of the whole blood, such as red blood cells (“RBCs”), platelets, plasma, or white blood cells (“WBCs”). Example apheresis systems 100 include SPECTRA OPTIA® apheresis system, COBE® spectra apheresis system, and/or TRIMA ACCEL® automated blood collection system, which are manufactured by Terumo BCT, of Lakewood, Colorado. The microfluidic device 150 is configured to process the extracted apheresis product 102 into precise components (or constituents). For example, in various aspects, the microfluidic device 150 may be configured to separate plasma, red blood cells, white blood cells, and/or platelets from the extracted apheresis product 102 for later use, including for different therapies, treatments, and/or transfusions. Example microfluidic devices 150 may include, for example, a means to purify a white blood cell product by removing red blood cell and platelet residuals from the original apheresis separation. The microfluidic device 150 may be used to wash and/or concentrate the apheresis product 102. Due to its precision geometry, which is on the same relative scale as blood cells, the microfluidic device 150 can perform, in various aspects, a precise separation of blood cells by size. For example, some reports indicate that the microfluidic separation can distinguish cell size differences down to a few microns. However, due to the size small yet precise geometry, it is often challenging to process large quantities of cells (such as is present in whole blood) using the microfluidic device 150 alone in a reasonable amount of time. The apheresis system 110, however, can easily process liters of whole blood into component, however, using the apheresis system 110 alone it can be difficult to achieve a high degree of precision, such as is capable when using the microfluidic device 150. The present disclosure provides means for using the apheresis system 100 and the microfluidic system 150 in combination.

In some example embodiments, as illustrated, a pressure device 170 may be disposed between the apheresis system 110 and the microfluidic device 150. For example, a first tube (or hose or other acceptable connector) 112 may fluidly connect the apheresis system 110 and the pressure device 170, and a second tube (or hose or other acceptable connector) 120 may fluidly connect the pressure device 170 and the microfluidic device 150. The first tube 112 may include a container (e.g., bag, vessel, bottle, etc.) 114 configured to temporarily collect and hold at least a portion of the extracted apheresis product 102. The first tube 112 may include, in addition to or in place of the container 114, one or more valves 116. For example, as illustrated, the first tube 112 may include a valve 116 disposed downstream of the container 114. In each instance, the one or more valves 116 may be configured stop and start fluid flow between the apheresis system 110 and the pressure device 170. Like the first tube 112, the second tube 120 may include one or more valves 122. The one or more valves 122 may be configured to stop and start fluid flow between the pressure device 170 and the microfluidic device 150. Although not illustrated, it should be recognized that the first tube 112 and/or the second tube 122 may be defined by one or more parts (or portions) connected, for example, using one or more sterile connectors.

The pressure device 170 may be configured to adjust the pressure of a container 180 (e.g., bag, vessel, bottle, etc.) prior to directing the container contents, or a portion thereof, to the microfluidic device 150. For example, the apheresis system 110 often delivers the extracted apheresis product 102 to a collection bag at normal or atmospheric pressure. The microfluidic device 150, however, requires much higher drive pressure (e.g., greater than or equal to about 70 psi to less than or equal to about 100 psi) to operate due to the inherently narrow cross-sectional area of its flow paths and high cellular velocity requirement, which are required to achieve the desired separation. Thus, in certain variations, the pressure device 170 may be configured to change the pressure of the apheresis product 102 (e.g., increase the pressure) prior to introducing the apheresis product 102 to the microfluidic device 150.

In various aspects, the container 180 may be configured to receive the extracted apheresis product 102 from the apheresis system 110, for example, via the first tube 112. The container 180 may also be configured to receive a buffer solution from 104 a buffer reserve (or container) 130. In certain variations, the buffer may include crystalloid solutions (e.g., isotonic saline) and/or solutions designed to enhance cellular storage. In each instance, the buffer may provide a fluid to suspend the cells sorted or separated by the microfluidic device 150. In certain variations, a third tube (or hose or other acceptable connector) 132 may fluidly connect the buffer reserve 130 and the container 180, and more particularly, the buffer reserve 130 and the second chamber 184 of the container 180. Like the first and second tubes, the third tube 130 may include one or more valves 134. The one or more valves 134 may be configured to stop and start fluid flow between the buffer reserve 130 and the pressure device 170. Further, although not illustrated, it should be recognized that the third tube 132 may be defined by one or more parts (or portions) connected, for example, using one or more sterile connectors.

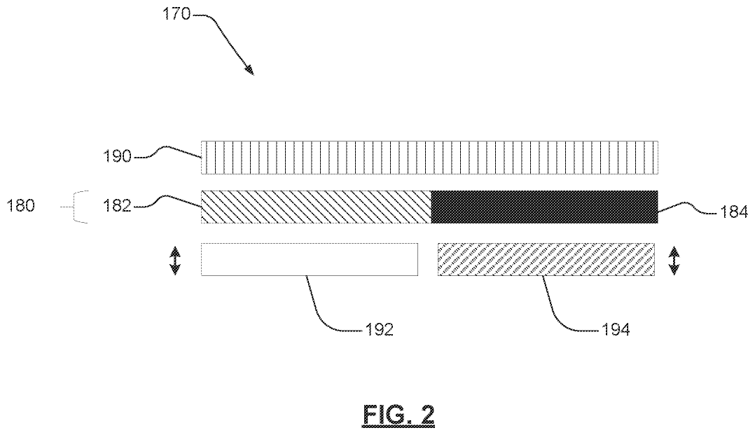

In certain variations, as illustrated in FIG. 2, the container 180 (e.g., high-pressure bag) may be segmented including one or more discrete areas or sections. For example, in certain variations, the container 180 includes a first chamber (or side or compartment) 182 and a second chamber (or side or compartment) 184. The first and second chambers 182, 184 may have that same or different sizes. A size ratio of the first chamber 182 to the second chamber 184 may be selected to cause a flow rate/volume ratio to be delivered to the microfluidic device 150. In certain variations, the second chamber 184 may be larger than the first chamber 182. For example, the second chamber 184 may be about three times larger than the first chamber 182. The difference in chamber size may provide a means by which to assure a volumetric or flow rate ratio between the cellular fluids 102 and the buffer fluids 104 which may be required by the microfluidic device 150. In each instance, the first chamber 182 may be configured to receive the extracted apheresis product 102, for example, via the first tube 112; and the second chamber 184 may be configured to receive the buffer solution 104, for example, from the buffer reserve (or container) 130, via the second tube 132. The movement of the extracted apheresis product 102 via the first tube 112 to the first chamber 182 and/or the movement of the buffer solution 104 via the third tube 132 to the second chamber 184 may occur using gravity fill processes.

As illustrated in FIG. 2, the pressure device 170 may include static (or stationary or reaction) plate 190 disposed on a first side 186 of the container 180 and one or more moveable plates 192, 194 disposed on a opposite (or second) side 188 of the container 180. For example, the container 180 may be disposed between the reaction plate 190 and first and second moveable plates 192, 194. A major axis of the reaction plate 190 may be substantially parallel with a major axis of the one or more moveable plates 192, 194, and the one or more moveable plates 192, 194 may be side-by-side, as illustrated. The first (or cell) moveable plate 192 may be associated (e.g., aligned) with the first chamber 182 of the container 180. The second (or buffer) moveable plate 194 may be associated (e.g., aligned) with the second chamber 184 of the container 180. Although not illustrated, in each variation, one or more drive motors may be configured to move the one or more moveable plates 192, 194 in a direction toward the static plate 190 and/or in a direction away from the static plate 190. When moved toward the static plate, the first moveable plate 192 and/or the second moveable plate 194 may press to the container 180 against the static plate 190 to change a pressure inside each chamber 182, 184 of the container 180. For example, in certain variations, the pressure device 170 may cause pressures of the first and second chambers 182, 184 to increase. In other variations, the pressure device may cause pressures of the first and second chambers 182, 184 to decrease. The one or more moveable plates 192, 194 may move together and/or independently using the same or different drive motors.

In some example embodiments, a controller is in electrical communication with the valve 116 and allows cells (e.g., extracted apheresis product 102) collected from the apheresis system 110 to enter the pressure device 170 (e.g., first chamber 182). The controller may also be in electrical communication with the valve 134 and allows buffer 104 to enter the pressure device (e.g., second chamber 184). Once a preselected amount of the extracted apheresis product 102 enters the pressure device 170, the controller may cause the valve 116 to close. Similarly, once a preselected amount of the buffer solution enters the pressure device 170, the controller may cause the valve 134 to close. Once the appropriate volumes are received, the controller may cause the container 180 to be subjected to a specific pressure, for example, as a result of the movement of the moveable plates 192, 194, such that a pressure inside the container 180 changes. The specific pressure may be greater or lower than a specific pressure of the container 180 prior to the receipt of the extracted apheresis product 102 and/or buffer solution 104.

With renewed reference to FIG. 1, one or more tubes (or hoses or other acceptable connectors) 140, 142 may fluidly connect the pressure device 170 and the microfluidic device 150. For example, as illustrated, a fourth tube (or hose or other acceptable connector) 140 may fluidly connect the first chamber 182 of the container 180 with the microfluidic device 150, and a fifth tube (or hose or other acceptable connector) 142 may fluidly connect the second chamber 184 of the container 180 with the microfluidic device 150. Like the first, second, and third tubes, the fourth tube 140 may include one or more valves 144. Similarly, the fifth tube 142 may include one or more valves 146. The one or more valves 144, 146 may be configured to stop and start fluid flow between the pressure device 170 and the microfluidic device 150. The one or more valves 144, 146 may be closed while the specific pressure of the container 180 is adjusted. Although not illustrated, it should be recognized that the fourth tube 140 and/or the fifth tube 142 may each be defined by one or more parts (or portions) connected, for example, using one or more sterile connectors.

While the first and second valves 116, 134 are closed, the third valve 144 and/or the fourth valve 146 may be opened. In this state, the controller may move the first moveable plate 192 and/or the second moveable plate 194, independently or together, in a direction toward the reaction plate 190 to apply a pressure to the container 180. When the pressure inside one or more of the chambers 182, 184 of the container 180 reaches a pre-selected specific pressure (e.g., about 70 pounds per square inch (psi)) the contents, or a portion thereof, in the container 180, may flow into the microfluidic device 150 via first and second ports (not shown) connected to the fourth tube 140 and/or fifth tube 142. The microfluidic device 150 may be in communication with a collection bag 160 and/or a waste bag 165. Although a microfluidic device 150 is discussed, it should be recognized that the any processing device (e.g., cell processing device) configured to receive materials at specific pressures may be used.

In various aspects, the present disclosure provides a method for using the incorporation systems, like the system 100 illustrated in FIG. 1. In some example embodiments, a method includes, for example, receiving cells (e.g., extracted apheresis product 102) collected during an apheresis procedure using an apheresis system or machine (like the apheresis system 110 illustrated in FIG. 1) in a pressurizable container (like container 180 illustrated in FIG. 2) that is connected to an apheresis system. The method also includes, during the apheresis procedure, adjusting a pressure inside the container to a pre-selected pressure using a pressure-generating device (like the pressure device 170 illustrated in FIG. 2). The pre-selected pressure may be associated with an in-line processing device (like the microfluidic device 150) that is connected with the container. The method also includes, during the apheresis procedure, transferring at least a portion of the cells from the container to the in-line processing device at the pre-selected pressure.

In some example embodiments, a method includes, for example, conveying cells (e.g., extracted apheresis product 102) collected during an apheresis procedure using an apheresis system or machine (like the apheresis system 110 illustrated in FIG. 1) to a first volume of a container (like container 180 illustrated in FIG. 2). The cells may be conveyed via a first fluid line (like fluid line 112 illustrated in FIG. 1) that fluidly connects the apheresis system to the container. The method also includes conveying a buffer solution to a second volume of the container. The buffer solution may be conveyed via a second fluid line (like fluid line 132 illustrated in FIG. 1) that fluidly connects a buffer source (like the buffer reserve 130 illustrated in FIG. 1) to the container. The method also includes closing a first valve of the first fluid line so as to block flow between the apheresis system and the container. The method also includes closing a second valve of the second fluid line so as to block flow between the buffer source and the container. The first and second valves may be opened and closed independently, including simultaneously or concurrently. The method also includes, after closing the first valve and/or second valve, opening a third valve that connects the container, and more particularly the first volume of the container, to a first port of a processing device (like the microfluidic device 150). The method also includes, after closing the first valve and/or second valve, opening a fourth valve that connects the container, and more particularly, the second volume of the container, to a second port of the processing device. The third and fourth valves may be opened and closed independently, including simultaneously or concurrently. The method also includes, after opening the third valve and/or the fourth valve, applying a pressure using a pressure-generating device (like the pressure device 170 illustrated in FIG. 2) to the container, including the first volume and/or the second volume, so that at least some of the contents of the container transfer to the processing device.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method for processing cells collected using an apheresis device, the method comprising:
receiving in a container the cells collected using the apheresis device;
adjusting a pressure of the container; and
at the adjusted pressure, transferring at least a portion of the cells collected using the apheresis device held by the container to a microfluidic device for processing.

2. The method of claim 1, wherein the adjusting of the pressure comprises decreasing the pressure inside the container from a first pressure to a second pressure.

3. The method of claim 1, wherein the adjusting of the pressure comprises increasing the pressure inside the consider form a first pressure to a second pressure.

4. The method of claim 3, wherein the first pressure is atmospheric pressure, and the second pressure is about 70 pounds per square inch (psi).

5. The method of claim 1, wherein the pressure is adjusted using a pressure system that is fluidly coupled to the apheresis device and the microfluid device, the pressure system comprising a stationary plate and one or more moveable plates that are configured to move relative to the stationary plate, wherein the container is disposed between the stationary plate and the one or more moveable plates and movement of the one or more moveable plates changes the pressure of the container.

6. The method of claim 5, wherein the container comprises a first compartment and a second compartment and the pressure system further comprises:
a first fluid line that fluidly connects the first compartment and the apheresis device, the first fluid line comprising a first valve;
a second fluid line that fluidly connects the second compartment and a buffer course, the second fluid line comprising a second valve;
a third fluid line that fluidly connects the first compartment and the microfluidic device, the third fluid line comprising a third valve; and
a fourth fluid line that fluidly connects the second compartment and the microfluidic device, the fourth fluid line comprising a fourth valve, and
wherein the method further comprises:
closing the first and second valves and opening the third and fourth valves prior to the adjusting of the pressure.
